(19) United States
(12) Patent Application Publication
(10) Pub. No.: US 2008/0015523 A1
(43) Pub. Date: Jan. 17, 2008

(54) MEDICAL AGENT DELIVERY SYSTEM AND METHOD

(75) Inventor: Randal S. Baker, Ada, MI (US)

Correspondence Address: VAN DYKE, GARDNER, LINN & BURKHART, LLP, SUITE 207, 2851 CHARLEVOIX DRIVE, S.E., GRAND RAPIDS, MI, 49546, US

(73) Assignee: SENTINEL GROUP, LLC, Grand Rapids, MI (US)

(21) Appl. No.: 11/780,050

(22) Filed: Jul. 19, 2007

Related U.S. Application Data
(63) Continuation-in-part of application No. PCT/US06/01654, filed on Jan. 18, 2006.
(60) Provisional application No. 60/645,487, filed on Jan. 19, 2005.

(30) Foreign Application Priority Data
Jan. 18, 2006 (US) PCT/US06/01654

(51) Int. Cl. A61M 31/00

(52) U.S. Cl. 604/288.01

(57) ABSTRACT

A medical agent delivery system and method of dispensing a medical agent include providing a medical agent dispensing member and supporting said medical agent dispensing member at the gastro-esophageal region of the patient.

### CROSS REFERENCE TO RELATED APPLICATIONS

This application is a continuation-in-part of International Application No. PCT/US06/01654, filed on Jan. 18, 2006, which claims priority from U.S. provisional patent application Ser. No. 60/645,487, filed on Jan. 19, 2005, the disclosures of which are hereby incorporated herein by reference in their entirety.

### BACKGROUND OF THE INVENTION

The present invention is directed to a technique for delivering a medical agent to a patient and, in particular, to a technique for time-release administration of an agent. The invention is not limited to any particular agent and may have application to dispensing a wide variety of substances. While the invention is illustrated for administering a therapeutic agent, such as a medication, it may also be used to administer a diagnostic agent, placebo, or the like.

Various techniques are available for administering an agent to a patient. In addition to traditional vascular access, such as subcutaneous and intramuscular injection, there are ingestible caplets and liquids as well as various skin patches. Intravascular stents are provided that have drug dispensing polymers that elude drugs into the blood stream. All of these delivery mechanisms have limitations. Present vascular access techniques may lead to complications, such as clotting, strictures and tightness of the vessel and infection. While skin patches are capable of time-release administration of a drug, they are not capable of adjustment of the dosage as a function of physical or chemical levels in the patient, such as glucose level, blood pressure, or the like. Also, intravascular stents require invasive procedures to implant and the medication is only for the purpose of avoiding occlusion of the stent, not for systemic dispensing of a medication. Also, they cannot be replenished in situ. Ingestible tablets and caplets are delivered to an acid environment in the stomach, which can have a deleterious affect on the agent being administered, thus limiting the agents that can be delivered in this manner.

Administration of certain blood-level-regulating drugs, such as diabetic medicines, requires monitoring of a chemical or physical level of the patient for feedback adjustment of the dosage administered. Because the monitoring typically occurs at infrequent intervals, it is possible to have wide swings in the blood level of the chemical being regulated. Also, natural insulin is secreted into the mesenteric system from the pancreas. Present modalities put the medicine into the vascular system where the first-pass effect of travelling, for example, through the liver, can reduce the therapeutic effect of the agent.

### SUMMARY OF THE INVENTION

The present invention is intended to deliver medical agents in a manner that mimics the natural functioning of the body. A medical agent delivery system and method of dispensing a medical agent, according to an aspect of the invention, includes providing an agent dispensing member and a support. The support is adapted to position the agent dispensing member at the gastro-esophageal region of a patient.

The agent dispensing member may include a replenishable agent reservoir and a diffusion member. The diffusion member dispenses an agent from the reservoir. The member may further include a fluid-receiving port. The port is in fluid connection with the agent reservoir. The port may be adapted to receive a blunt needle, such as a blunt needle that is inserted endoscopically. The port may be made up of flexible-connection tubing, such as one that is adapted to terminate subcutaneously.

The support may have a wall that is configured to generally conform to the size and shape of the abdominal portion of the esophagus, the esophageal-gastric junction and/or the proximal cardiac portion of the stomach. At least one fixation mechanism may be provided that is adapted to resist distal migration of the support. The fixation mechanism may include barbs, V-shaped appendages, metal anchors extending regularly from the body, staples, or sutures. Alternatively, the fixation mechanism may include an inflatable anchor bladder. Alternatively, the fixation mechanism may include a portion of the wall having natural tissue ingrowth orifices. The wall may have a generally cylindrical portion and a generally conical portion at least part of which is expandable. The agent dispensing member may be adapted to dispense the agent at the generally conical portion of the wall.

The agent dispensing member may include a tissue interface. The tissue interface may include a diffusion member that is adapted to dispense an agent to the muscularis, the mucosa, or the sub-mucosa. The diffusion member may be made up of a semi-permeable membrane that at least partially enclosed an agent reservoir.

The medical delivery system may further include a control that controls a rate at which the dispensing member dispenses an agent. The control may include a sensor. The control controls the rate at which the dispensing member dispenses a drug as a function of an output of the sensor. The support may include a wall having a conical portion that is configured to conform to the size and shape of the proximal cardiac portion of the stomach. The sensor may be positioned to sense at the general conical portion. The sensor may include a tissue contact that is adapted to sense at least one parameter at a portion of the proximal cardiac portion of the stomach. This portion may include the muscularis, the mucosa or the sub-mucosa.

The sensor may sense a chemical and/or a physical parameter of the patient. The control may transfer an agent from a reservoir to a diffusion member as a function of an output of the sensor. The medical agent delivery system may include a remote controller and a wireless communication link between the remote controller and the control whereby the remote controller is adapted to adjust the control. The control may include a microchip.

The agent dispensing member may be adapted to dispense an agent to the stomach cavity. The diffusion member may be a semi-permeable membrane. The agent-dispensing member may be a time-release polymer.

These and other objects, advantages and features of this invention will become apparent upon review of the following specification in conjunction with the drawings.

### BRIEF DESCRIPTION OF THE DRAWINGS

FIG. 1 is a diagram of a medical agent delivery system positioned at the gastro-esophageal portion of the patient;

FIG. 2 is the same view as FIG. 1 illustrating an alternative embodiment thereof;

FIG. 3 is the same view as FIG. 1 illustrating another alternative embodiment thereof;

FIG. 4 is the same view as FIG. 1 illustrating another alternative embodiment thereof;

FIG. 5 is the same view as FIG. 1 illustrating another alternative embodiment thereof;

FIG. 6 is the same view as FIG. 1 illustrating another alternative embodiment thereof;

FIG. 7 is the same view as FIG. 1 illustrating another alternative embodiment thereof;

FIG. 8 is a block diagram illustrating details of a medical agent delivery member;

FIG. 9 is the same view as FIG. 8 of an alternative embodiment thereof;

FIG. 10 is the same view as FIG. 8 of another alternative embodiment thereof; and

FIG. 11 is the same view as FIG. 8 of another alternative embodiment thereof.

### DESCRIPTION OF THE PREFERRED EMBODIMENT

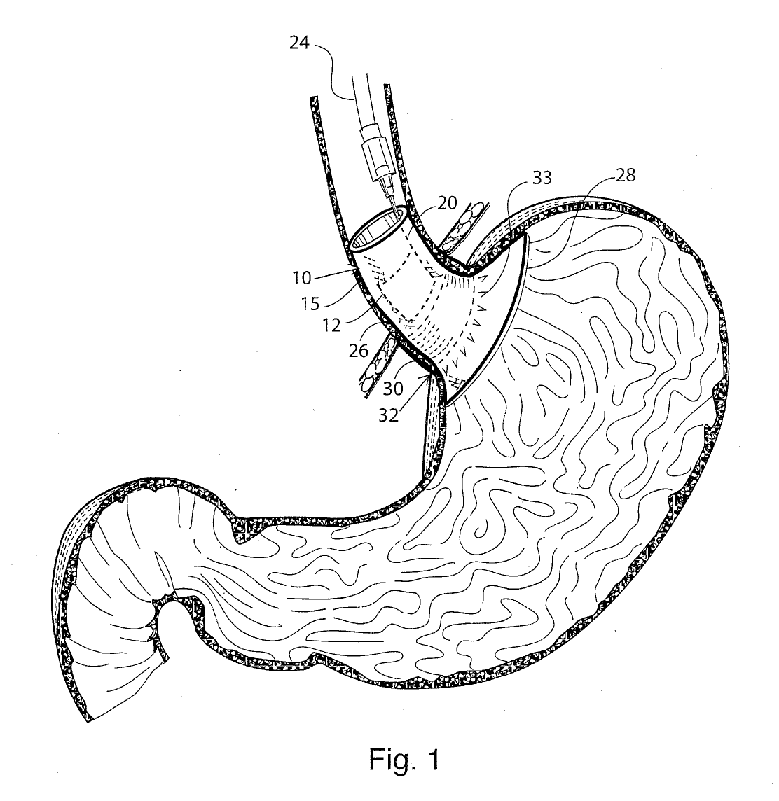

Referring now specifically to the drawings, and the illustrative embodiments depicted therein, a medical agent delivery system 10 includes an agent dispensing member 12 and a support 14 (FIG. 1). Support 14 includes a wall 15 that is configured to position agent dispensing member 12 at the gastro-esophageal region of the patient. This may include the esophagus or upper stomach, in general, or the abdominal portion of the esophagus, the esophageal gastric junction, or the cardia, in particular. In certain of the illustrative embodiments, support 14 is made up of a bariatric device of the type disclosed in Patent Cooperation Treaty Application Ser. No. PCT/US2005/036991 filed Oct. 13, 2005, entitled BARIATRIC DEVICE AND METHOD, by Baker et al., the disclosure of which is hereby incorporated herein by reference in its entirety. Other supports may be used, as will be disclosed in detail below.

As disclosed in the Baker et al. patent application previously referred to, support 14 may have a first portion 26 configured to the distal esophagus, or abdominal portion of the esophagus, of the patient and a portion 28 configured to engage the wall at the cardia of the patient. Esophageal portion 26 is generally cylindrical in shape, and cardia portion 28 is generally conical in shape. Portions 26 and 28 may be expandable, such as self-expanding, in order to exert radial pressure on the distal esophagus and cardia portion of the stomach. A central portion 30 between the esophageal portion and the cardia portion may be made from a flaccid material. The central portion, when positioned at the esophageal sphincter, allows normal functioning of the esophageal sphincter. This allows belching, vomiting, and the like, to occur naturally as well as allows the natural anti-reflux mechanism of the body to operate normally. Support 14 additionally includes a fixation mechanism, generally shown at 32, to resist distal migration of support 14. Fixation mechanism 32 may include V-shaped appendages 33 for anchoring the support. Other fixation mechanisms may include barbs, hooks, metal anchors extending radially from support 14, suture or staples. Fixation mechanism 32 may be in the form of a wall 15 of support 14 including an inflatable bladder (not shown) to expand wall 15 outwardly. In the embodiment illustrated in FIG. 1, fixation mechanism 32 includes natural tissue ingrowth orifices defined on a portion of wall 15, such as central portion 30. The tissue ingrowth orifices allow tissue ingrowth to resist distal migration. They may be used in combination with other fixation mechanisms, such as biodissolvable sutures, staples, or the like, to retain the support during tissue ingrowth. Other anti-migration structures that would be apparent to those skilled in the art may be used. While the invention is illustrated with certain embodiments of the bariatric device illustrated in the Baker et al. patent application previously referred to, it is not intended that the invention be limited to embodiments disclosed in the Baker et al. patent application.

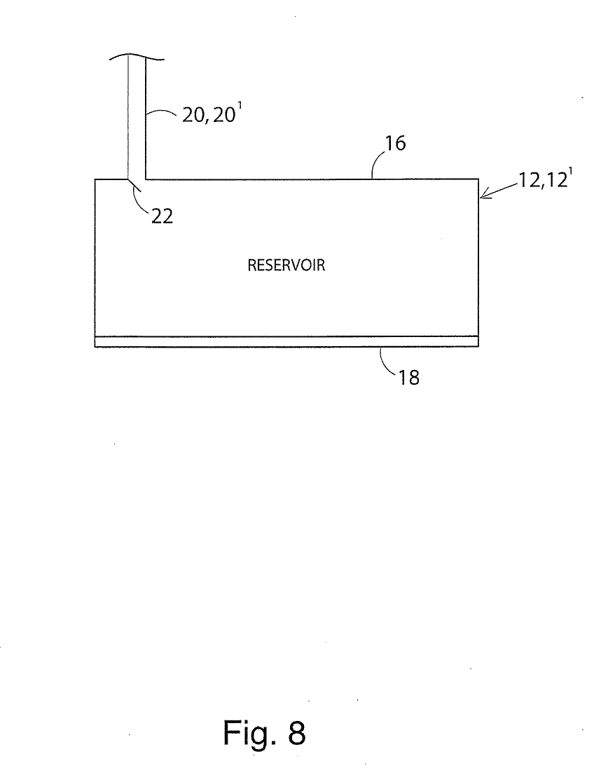

Agent dispensing member 12 may include a replenishable agent reservoir 16 and a diffusion member 18 dispensing the agent from reservoir 16 (FIG. 8). The physical characteristics of diffusion member 18 may influence the release rate of the agent. Diffusion member 18 in the illustrative embodiment is a semi-permeable membrane which allows diffusion of the drug into the stomach wall, cavity, or the like. As will be discussed in more detail below, the agent may be released into the stomach cavity, either directly or via the esophagus, or may be applied to the stomach wall. By application of the agent to the stomach wall by way of the muscularis, the mucosa and/or the sub-mucosa, the agent may be applied directly to the mesenteric vascular bed as will be described in more detail below.

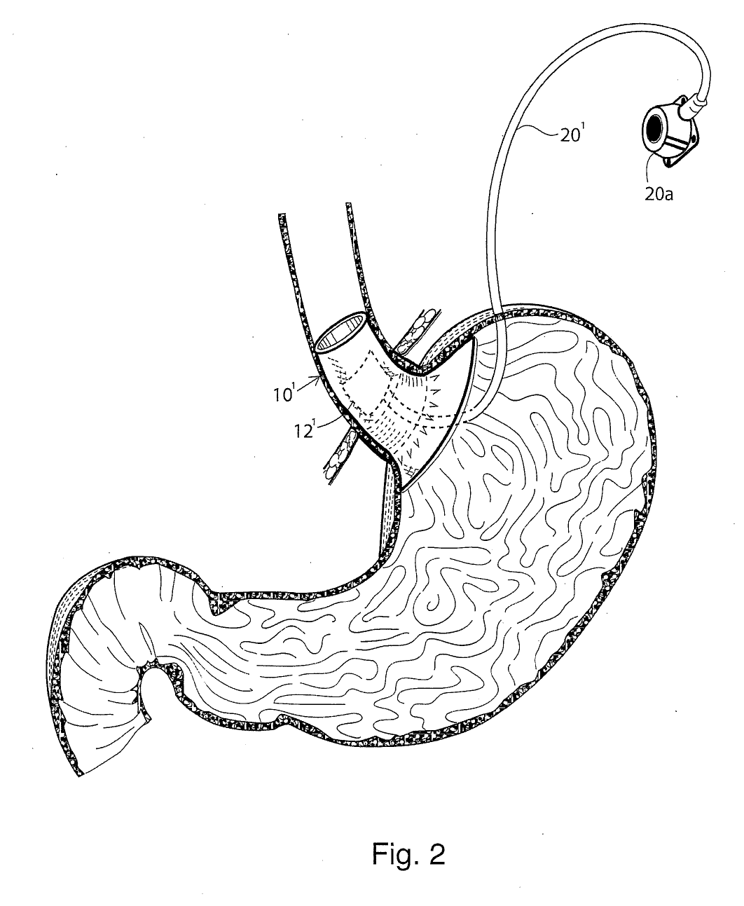

Agent dispensing member 12 may include a port 20 in fluid connection with reservoir 16 in order to replenish the drug in reservoir 16. A one-way valve 22 may be used to ensure that the drug in reservoir 16 does not exit through port 20. As illustrated in FIG. 1, port 20 may be configured to receive a blunt needle 24 that is inserted into port 20. The blunt needle may be inserted endoscopically with fluoroscopic assist. Alternatively, as illustrated in FIG. 2, a medical agent delivery system 10′ may include an alternative agent dispensing member 12′ having a port 20′ that extends through the wall of the stomach to a port 20a at a subcutaneous portion of the patient. Such a subcutaneous port is well known in the art and may be accessed through the skin. Other techniques may be apparent to those skilled in the art for replenishing reservoir 16.

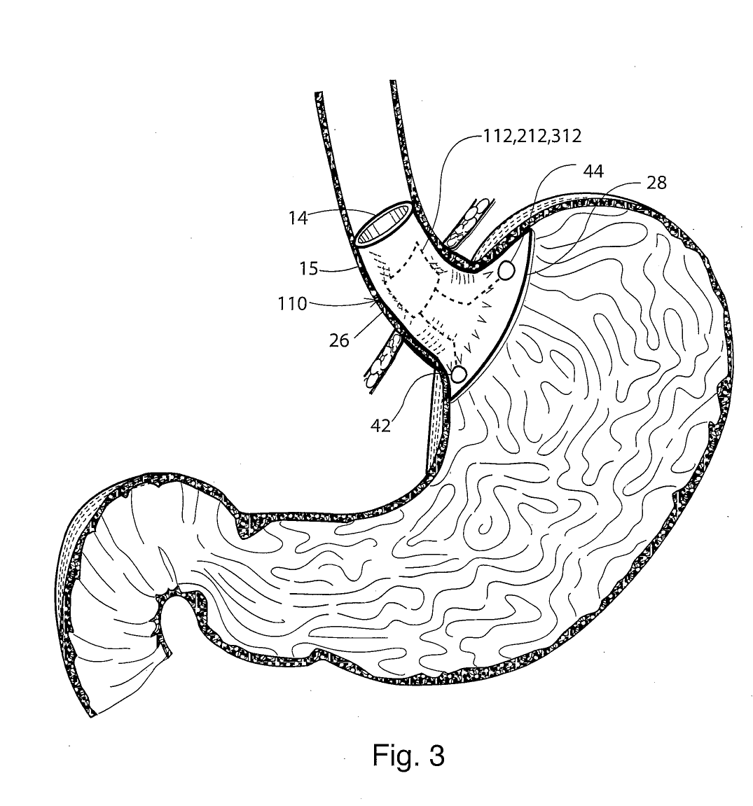

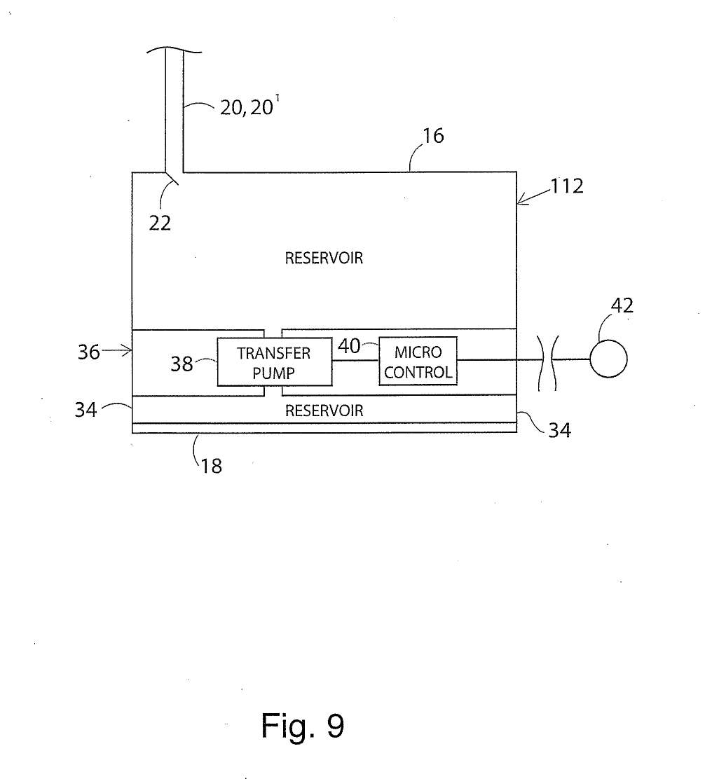

In an alternative embodiment, a medical agent delivery system 110 includes a medical agent dispensing member 112, 212, 312 and a support 114 for supporting the agent dispensing member within the gastro-esophageal portion of the patient (FIGS. 3 and 9-11). Agent dispensing member 112, 212, 312 may include a dispensing reservoir 34 in fluid communication with diffusion member 18. Agent dispensing member 112, 212, 312 may additionally include a transfer mechanism 36 for transferring fluid between storage reservoir 16 and dispensing reservoir 34. Transfer mechanism 36 may include a microtransfer pump 38, a valve, or the like, and a microcontroller 40. Microcontroller 40 controls the rate of transfer of the drug by transfer pump 38. In order to increase the rate of dispensing of the drug, transfer mechanism 36 transfers the agent from reservoir 16 to reservoir 34 at a higher rate and decreases the rate of dispensing by transferring the agent at a lower rate.

Agent dispensing member 112, 212, 312 may additionally include a sensor 42 for providing a feedback mechanism to operate microcontrol 40 in a feedback loop. Sensor 42 senses a parameter of the patient, such as the chemical level of the blood, or a physical parameter, such as blood pressure, stomach pH, or the like. Sensor 42 may be in the form of a tissue contact that is configured to interconnect with a wall of the gastro-esophageal region, such as the stomach wall. Sensor 42 may contact the muscularis, the mucosa and/or the sub-mucosa of the stomach wall. In the illustrated embodiment, sensor 42 is positioned on cardiac portion 28 of wall 15, as illustrated in FIG. 3. Cardiac portion 28 presses sensor 42 against the stomach wall to provide adequate contact because of the expandable nature of the cardia portion.

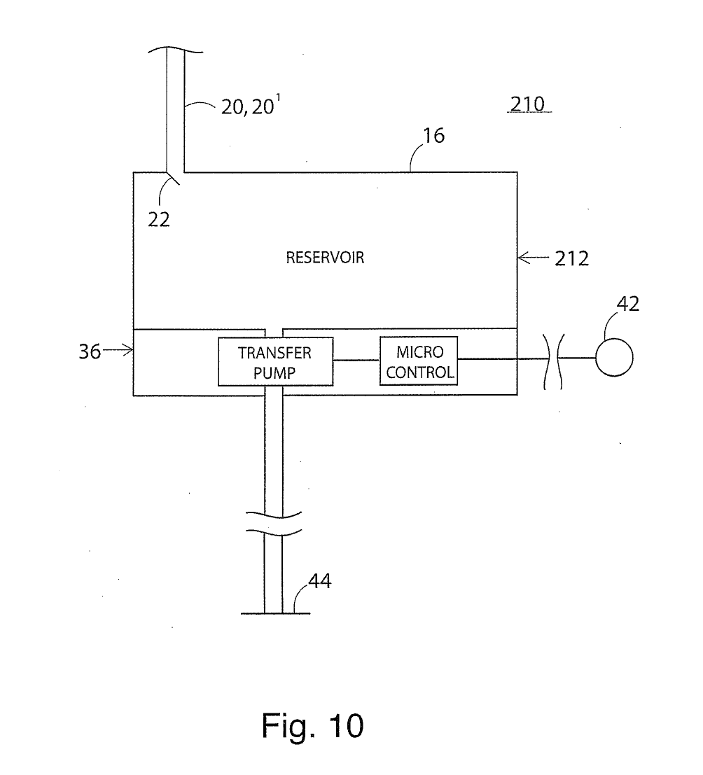

In another alternative embodiment, a medical agent dispensing member 212 is provided that includes a diffusion member in the form of a tissue interface 44 that is configured to dispense the agent to the stomach wall (FIG. 10). Tissue interface 44 may dispense the agent to the muscularis, the mucosa, and/or the sub-mucosa of the stomach wall. Conveniently, tissue interface 44 may be disposed on the cardia portion 28 of wall 15. Cardia portion 28 positions tissue interface 44 in contact with the stomach wall because of the expandable nature of the cardia portion.

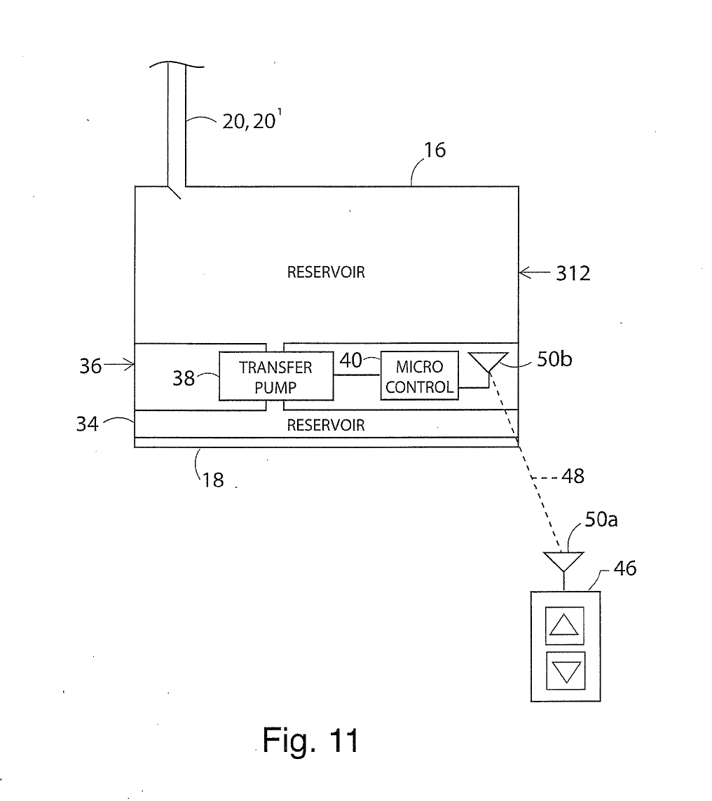

In another alternative embodiment illustrated in FIG. 11, an agent dispensing member 312 is provided in which microcontroller 40 is controlled by a control unit 46 that is external to the patient. Control unit 46 communicates with microcontrol 40 by way of a wireless connection, such as a radio-frequency link 48 between an antenna 50a on the control unit and 50b on the microcontroller internal to the drug dispensing member. This allows the rate of dispensing of the drug to be controlled external to the patient. Also, microcontroller 40 may communicate with control unit 46 over a radio-frequency link 48 to send status information, such as a low drug level warning, and the like.

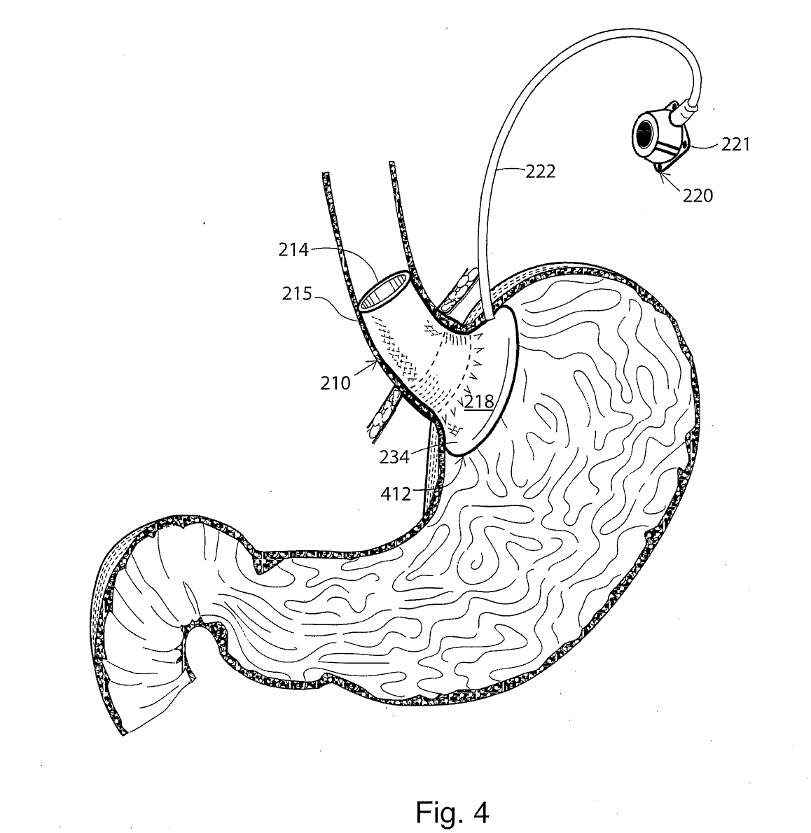

In another embodiment illustrated in FIG. 4, a medical agent delivery system 210 includes an agent dispensing member 412 that is supported by, or formed integrally with wall 215 of support 214. Medical agent dispensing member 412 includes a dispensing reservoir 234 defined, in part, by a diffusion member 218 in the form of a semi-permeable membrane. Dispensing reservoir 234 may be otherwise partially formed by a non-diffusion member. If diffusion member 218 faces toward the stomach wall, then member 218 forms a tissue interface. This allows diffusion member 218 to dispense the agent to the stomach wall and, hence, the muscularis, the mucosa and/or the sub-mucosa. If diffusion member 218 faces away from the stomach wall, then diffusion member 218 is capable of dispensing the agent to the contents of the stomach. A combination of the two is also possible.

Diffusion member 218 may additionally incorporate tissue ingrowth surface portions into the diffusion member that defines the tissue interface. The tissue ingrowth surface portions may be formed as a pattern that is mixed in with the semi-permeable membrane. Also, the tissue ingrowth surface portions may be septations that extend from the surface of diffusion member 218. In this manner, a ratio of semi-permeable surface area to tissue ingrowth surface area may be configured to provide an increased surface contact area with the mucosa for enhanced bio-absorbability of the medical agent.

In particular, the combination of tissue ingrowth surface portions and semi-permeable surface portions is capable of providing enhanced mucosal coverage of the diffusion surface ans increased vascularization of the portion of the stomach in contact with the diffusion surface to enhance absorbability of the medical agent in the vascular beds of the mesenteric system. Also, the tissue ingrowth surface portions enhance anchoring of the medical agent delivery system at the gastro-esophageal region of the patient defined by the esophageal-gastric junction, the abdominal portion of the esophagus, and/or other portions of the stomach.

The combination of tissue ingrowth surface portions and semi-permeable surface portions may be configured to induce mucosa coverage to the portion of the diffusion surface facing away from the stomach wall as well as the portion facing the stomach wall to further enhance mucosal coverage.

Medical agent delivery system 210 may include a port 220 in the form of a subcutaneous access member 221 and a flexible connection tubing 222 passing through the stomach wall. Subcutaneous access member 221 may include a storage reservoir, pump, and microcontroller (not shown). The agent in the storage reservoir can be replenished subcutaneously. The microcontroller controls the rate that the agent is pumped from the storage reservoir to dispensing reservoir 234 through connection tubing 222 and thereby controls the rate that the agent is dispensed to the patient. The positioning of the storage reservoir, pump, and microcontroller at the subcutaneous access member reduces the weight and bulk of the items supported by support 214. Alternatively, subcutaneous access member 221 may allow manual addition of an agent to dispensing reservoir 234, such as by a syringe, or the like.

Medical agent delivery system 210 may include a sensor (not shown) for providing a feedback mechanism to operate the microcontroller in port 220. The sensor may be positioned on agent dispensing member 412, such as in contact with the stomach wall. The sensor may be interconnected with the microcontroller, such as by wires running along or within tubing 222 by a wireless communication channel, or the like.

Thus, it is seen that the present invention provides a unique drug delivery system that overcomes many of the difficulties in previous devices. The system can be inserted and removed endoscopically with fluoroscopic assist. The reservoir can be refilled in a relatively non-intrusive manner, such as endoscopically, through a subcutaneous port, or the like. Because the stomach wall has extensive neuro and hormonal connections, blood chemical levels can be readily monitored in order to regulate the levels of chemicals in the blood. This can be done on an essentially real-time basis, thereby reducing peaks and valleys in important blood levels, such as glucose, and the like. Also, agents can be effectively delivered to the bloodstream through the stomach wall with its rich vascular bed.

One particular agent for which the present invention is particularly useful is the delivery of diabetic medicine, such as hypoglycemics and insulin. The pancreas delivers natural insulin to the mesenteric system. In known delivery modalities, the agent is put into the vascular system where it passes first through other organs, such as the liver, before it reaches the mesenteric system. This may create a first-pass effect, whereby the effectiveness of the agent is reduced before it is delivered where it is required. In contrast, an agent delivery system, according to the invention, delivers the agent to the vascular beds of the mesenteric system surrounding the stomach. This avoids the first-pass effect of known modalities. Also, because the diabetic medicine is delivered to the stomach wall and not to the stomach contents, the effect of stomach acid on the medicine is precluded.

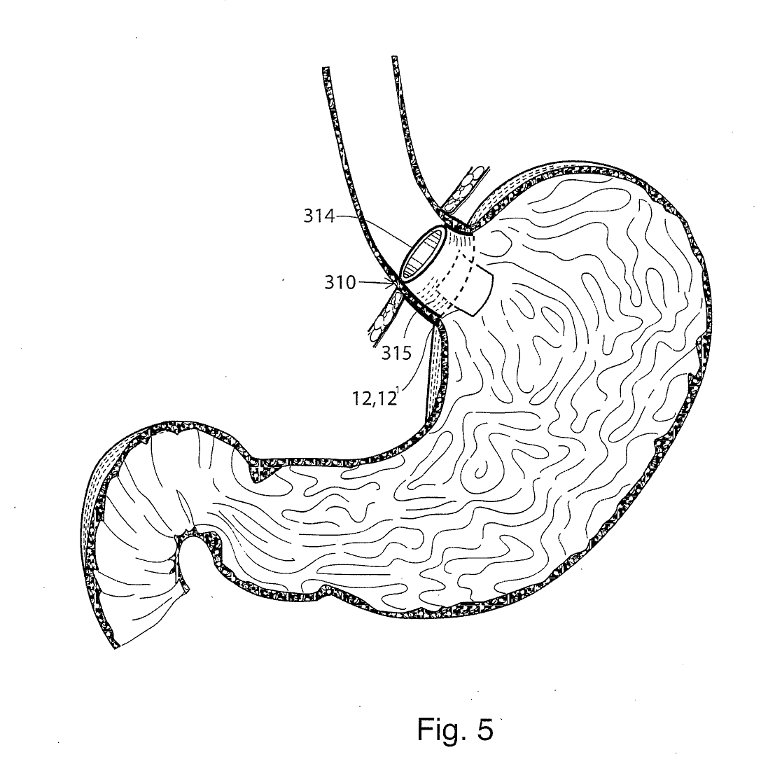

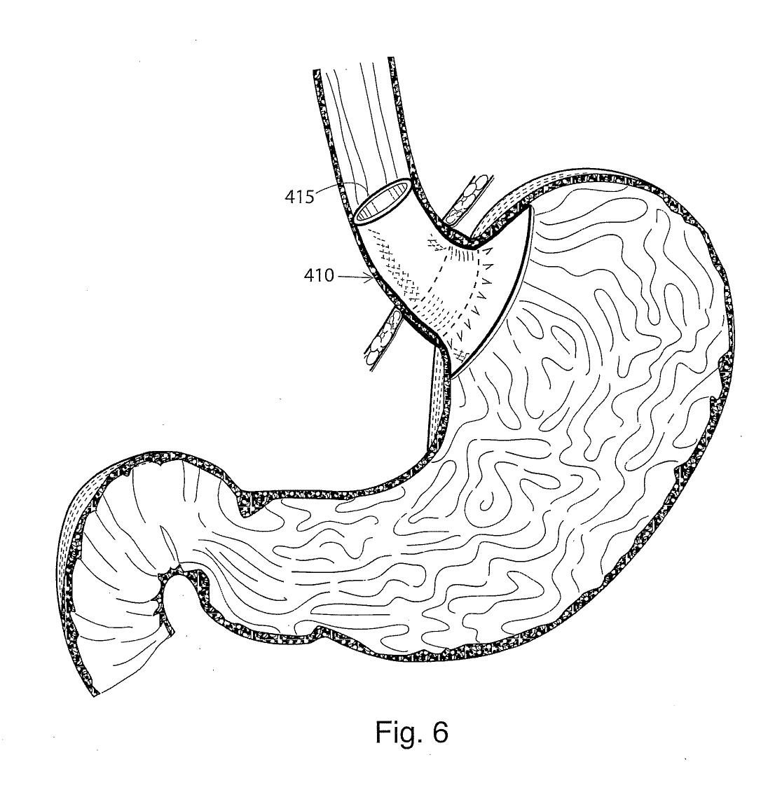

While various embodiments of the invention are illustrated herein, it should be understood that various combinations of embodiments would be apparent to those skilled in the art. For example, an agent delivery system 310 is illustrated in FIG. 5, which includes a support 314 having a wall 315 covered all or in part by natural tissue ingrowth orifices. This allows support 314 to support a medical agent dispensing member 12, 12′ without the necessity of applying outward pressure on any portion of the gastro-esophageal region of the patient. Support 314 resists distal migration by the ingrowth of tissue through the natural tissue ingrowth orifices. In an alternative embodiment illustrated in FIG. 6, a medical agent delivery system 410 is defined by a wall 415 which incorporates a time-release polymer of the type known in cardiovascular drug eluding devices, while wall 415 may also be made of a bioabsorbable material. In this manner the wall and its agent may dissolve over time thereby eliminating the necessity for removal of the agent delivery system. Medical agent delivery system 410 would be replaced, rather than refilled, if necessary.

Other anchoring devices and methods may be used to avoid distal migration such as the techniques disclosed in pending patent application Ser. Nos. 60/901,457, filed Feb. 14, 2007, by Baker et al., entitled BARIATRIC DEVICE AND METHOD; 60/921,930, filed Apr. 5, 2007, by Baker et al., entitled BARIATRIC DEVICE AND METHOD; and 60/931,109, filed May 21, 2007, by Baker, entitled BARIATRIC DEVICE AND METHOD, the disclosures of which are hereby incorporated herein by reference in their entirety.

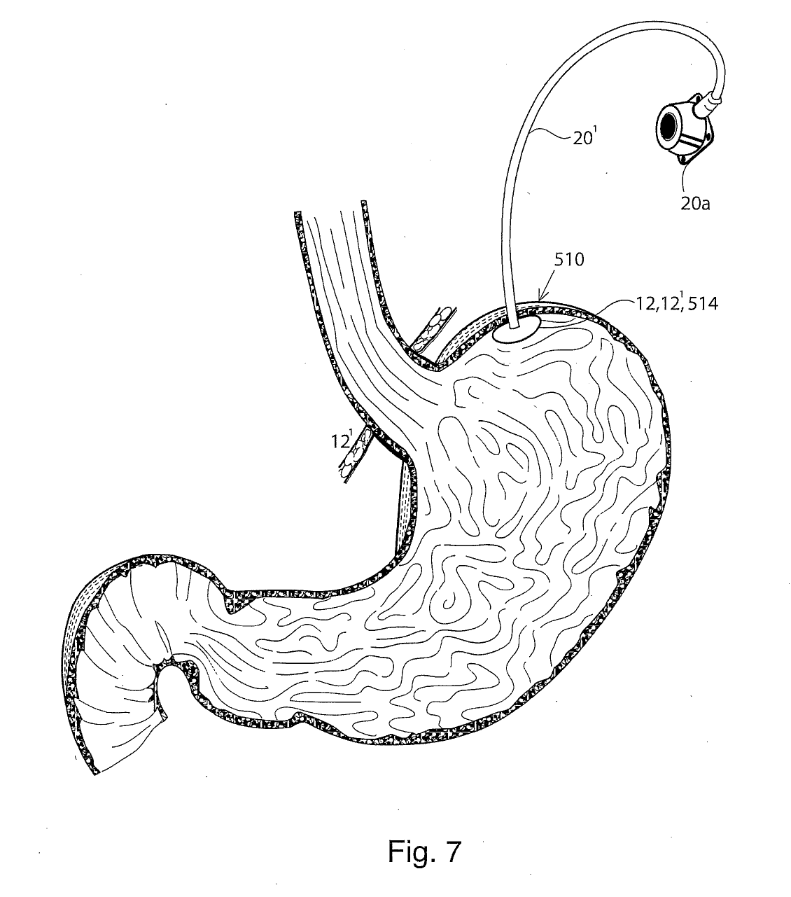

A medical agent delivery system 510, illustrated in FIG. 7, includes a support 514 that supports a medical agent dispensing member 12, 12′ at the cardiac portion of the stomach. A port 20′ facilitates replenishment of the agent from a subcutaneous port 20a. Medical agent delivery system 510 is positioned entirely outside of the patient's esophagus. Support 514 may include a fixation system, such as previously described. Alternatively, support 514 may support a medical agent dispensing member that incorporates a time-release polymer thereby not requiring a supply port.

The agent dispensing member disclosed herein is capable of dispensing a wide variety of therapeutic agents as well as other agents, such as diagnostic agents. Without limitation, examples of agents that may be dispensed include:
a) Pain medications
b) Chemotherapeutic agents
c) Antibiotic/antifungal agents
d) Antidepressants
e) Antisecretory medicines
f) Contraceptive agents
g) Diabetic medicines, such as hypoglycemics and insulin
h) Lipid-lowering medications
i) Antihypertensive medications
j) Gastric/bowel stimulant medications
k) Antipsychotic agents
l) Flavored breath freshening solutions
m) Antispasmodic medications
n) Vitamins and minerals
o) Placebos

Changes and modifications in the specifically described embodiments can be carried out without departing from the principles of the invention which is intended to be limited only by the scope of the appended claims, as interpreted according to the principles of patent law including the doctrine of equivalents.

The embodiments of the invention in which an exclusive property or privilege is claimed are defined as follows:

1. A medical agent delivery system, comprising:
a medical agent dispensing member; and
a support supporting said agent dispensing member, said support adapted to position said agent dispensing member at the region of a patient defined by at least one chosen from (i) the abdominal portion of the esophagus, (ii) the esophageal-gastric junction, and (iii) the cardiac portion of the stomach.

2. The medical agent delivery system as claimed in claim 1 wherein said agent dispensing member comprises a replenishable agent reservoir and a diffusion member, said diffusion member dispensing an agent from said reservoir.

3. The medical agent delivery system as claimed in claim 2 including a fluid receiving port, said port in fluid connection with said agent reservoir.

4. The medical agent delivery system as claimed in claim 3 wherein said port is adapted to receive a blunt needle.

5. The medical agent delivery system as claimed in claim 4 wherein said port is adapted to receive a blunt needle inserted endoscopically.

6. The medical agent delivery system as claimed in claim 3 wherein said port comprises a flexible-connection tubing.

7. The medical agent delivery system as claimed in claim 6 wherein said tubing is adapted to terminate subcutaneously.

8. The medical agent delivery system as claimed in claim 1 wherein said support has a wall configured to generally conform to the size and shape of at least one chosen from (i) the abdominal portion of the esophagus, (ii) the esophageal-gastric junction and (iii) the cardiac portion of the stomach.

9. The medical agent delivery system as claimed in claim 8 including at least one fixation mechanism that is adapted to resist distal migration of said support.

10. The medical agent delivery system as claimed in claim 9 wherein said fixation mechanism includes at least one chosen from barbs, V-shaped appendages, metallic anchors extending radially from said body, staples and sutures.

11. The bariatric device as claimed in claim 9 wherein said fixation mechanism includes an inflatable anchor bladder.

12. The bariatric device as claimed in claim 9 wherein said fixation mechanism includes at least a portion of said wall having natural tissue ingrowth orifices.

13. The medical agent delivery system as claimed in claim 8 wherein said wall has a generally cylindrical portion and a generally conical portion.

14. The medical agent delivery system as claimed in claim 8 wherein at least part of said generally cylindrical portion and said generally conical portion are expandable.

15. The medical agent delivery system as claimed in claim 13 wherein said agent dispensing member is adapted to dispense the agent at said generally conical portion.

16. A medical agent delivery system, comprising:
a medical agent dispensing member; and
a support supporting said agent dispensing member, said support adapted to position said agent dispensing member at the gastro-esophageal region of a patient;
wherein said agent dispensing member comprises a tissue interface, said tissue interface adapted to dispense an agent to at least one chosen from the muscularis, the mucosa and the sub-mucosa; and
wherein said agent dispensing member further comprises an agent reservoir and a port, said port adapted to provide access to said reservoir from external the patient.

17. The medical agent delivery system as claimed in claim 16 wherein said tissue interface comprises a diffusion member adapted to engage the at least one chosen from the muscularis, the mucosa, and the sub-mucosa.

18. The medical agent delivery system as claimed in claim 17 wherein said diffusion member comprises a semi-permeable membrane at least partially enclosing an agent reservoir.

19. The medical agent delivery system as claimed in claim 16 including a control, said control controlling a rate at which said dispensing member dispenses an agent.

20. The medical agent delivery system as claimed in claim 19 wherein said control includes a sensor, said control controlling the rate at which said dispensing member dispenses an agent as a function of an output of said sensor.

21. The medical agent delivery system as claimed in claim 20 wherein said support includes a wall having a conical portion that is configured to conform to the size and shape of the cardiac portion of the stomach and wherein said sensor senses at said generally conical portion.

22. The medical agent delivery system as claimed in claim 20 wherein said sensor comprises a tissue contact, said tissue contact adapted to sense at least one parameter at a portion of the cardiac portion of the stomach, said portion chosen from the muscularis, the mucosa and the sub-mucosa.

23. The medical agent delivery system as claimed in claim 20 wherein said sensor senses at least one chosen from a chemical level and a physical parameter of the patient.

24. The medical agent delivery system as claimed in claim 19 wherein said control transfers the agent from a reservoir to a diffusion member as a function of an output of said sensor.

25. The medical agent delivery system as claimed in claim 19 including a remote controller and a wireless communication link between said remote controller and said control whereby said remote controller is adapted to adjust said control.

26. The medical agent delivery system as claimed in claim 19 wherein said control comprises a microchip.

27. The medical agent delivery system as claimed in claim 1 wherein said agent dispensing member is adapted to dispense an agent to the stomach cavity.

28. The medical agent delivery system as claimed in claim 2 wherein said diffusion member comprises a semi-permeable membrane.

29. The medical agent delivery system as claimed in claim 1 wherein said agent dispensing member comprises a time-release polymer.

30. The medical agent delivery system as claimed in claim 1 wherein said agent dispensing member and said support are made from bioabsorbable materials.

31. A medical agent delivery system, comprising:
a medical agent dispensing member; and
a support supporting said agent dispensing member, said support adapted to position said agent dispensing member at the gastro-esophageal region of a patient;
wherein said agent dispensing member comprises a tissue interface, said tissue interface adapted to dispense an agent to at least one chosen from the muscularis, the mucosa and the sub-mucosa; and
wherein said tissue interface includes at least one tissue ingrowth surface portion.

32. The medical agent delivery system as claimed in claim 31 wherein said tissue interface comprises a combination of said at least one tissue ingrowth surface portion and at least one semi-permeable membrane portion.

33. A method of dispensing a medical agent, comprising:
providing a medical agent dispensing member and a support; and
supporting said medical agent dispensing member with said support at the region of a patient defined by at least one chosen from (i) the abdominal portion of the esophagus, (ii) the esophageal-gastric junction, and (iii) the cardiac portion of the stomach.

34. A method of dispensing a medical agent, comprising:
providing a medical agent dispensing member and a support;
supporting said medical agent dispensing member with said support at the gastro-esophageal region of a patient;
wherein said agent dispensing member comprises an agent reservoir, a tissue interface and a port;
dispensing an agent from said agent reservoir with said tissue interface to at least one chosen from the muscularis, the mucosa and the sub-mucosa; and
accessing said agent reservoir with said port from external the patient.

35. A method of dispensing a medical agent, comprising:
providing a medical agent dispensing member and a support;
supporting said medical agent dispensing member with said support at the gastro-esophageal region of a patient;
wherein said agent dispensing member comprises a tissue interface wherein said tissue interface includes at least one tissue ingrowth surface portion; and
dispensing an agent from said agent reservoir with said tissue interface to at least one chosen from the muscularis, the mucosa and the sub-mucosa.
